(19) United States
(12) Patent Application Publication
(10) Pub. No.: US 2017/0135918 A1
(43) Pub. Date: May 18, 2017

(54) Dentifrice Compositions With Whitening Benefit

(71) Applicant: The Procter & Gamble Company, Cincinnati, OH (US)

(72) Inventors: Swapna Basa, Beijing (CN); Hongmei Yang, Beijing (CN); Ross Strand, Singapore (SG)

(21) Appl. No.: 15/347,837

(22) Filed: Nov. 10, 2016

(30) Foreign Application Priority Data
Nov. 13, 2015 (WO) CN2015/094515

(51) Int. Cl. A61K 8/24; A61Q 11/02; A61Q 11/00; A61K 8/21; A61K 8/19

(52) U.S. Cl. CPC A61K 8/24; A61K 8/21; A61K 8/19; A61Q 11/00; A61Q 11/02

(57) ABSTRACT

A dentifrice composition containing water, a calcium-containing abrasive, a fluoride ion source, and an ultraphosphate where the composition can have tooth whitening benefits.

### FIELD OF THE INVENTION

The present invention relates to certain dentifrice compositions having tooth whitening benefits.

### BACKGROUND OF THE INVENTION

Dentifrice compositions are well known for dental and oral hygiene care. High water (e.g., >44 wt %) and high carbonate (e.g., >24 wt %) formulation chassis are cost effective for many markets and consumers. These compositions are formulated at relatively high pH (e.g., pH 8-11) for many reasons including fluoride stability (e.g., sodium monofluorophosphate). Tooth whitening benefits from dentifrice compositions are desired benefit. However, finding an effective tooth whitening agent that is effective in these dentifrice compositions remains a challenge. Accordingly, there is a need to identify a tooth whitening agent for a high water (e.g., >44 wt %), high carbonate (e.g., >24 wt %), alkaline dentifrice compositions.

### SUMMARY OF THE INVENTION

A surprising discovery is the use of ultraphosphate in high water, high carbonate, fluoride ion source, alkaline dentifrice formulations to provide tooth whitening benefits. One aspect of the invention provides for a dentifrice composition comprising: 45% to 75%, by weight of the composition, of water; 25% to 50%, by weight of the composition, of a calcium-containing abrasive (e.g., calcium carbonate); 0.0025% to 2%, by weight of the composition, of a fluoride ion source (e.g., sodium monofluorophosphate); (d) 0.4% to 10%, by weight of the composition, of an ultraphosphate; and wherein said composition has a pH greater than 8.

Yet another aspect of the invention provides a method of treating tooth enamel comprising the step of brushing teeth with a dentifrice composition of the present invention.

Yet still another aspect of the invention provides a method preventing and/or removing a stain from teeth comprising the step of brushing teeth with a dentifrice composition of the present invention.

These and other features, aspects, and advantages of the present invention will become evident to those skilled in the art from the detailed description which follows.

### DETAILED DESCRIPTION OF THE INVENTION

### Definitions

The term “comprising” as used herein means that steps and ingredients other than those specifically mentioned can be added. This term encompasses the terms “consisting of” and “consisting essentially of.” The compositions of the present invention can comprise, consist of, and consist essentially of the essential elements and limitations of the invention described herein, as well as any of the additional or optional ingredients, components, steps, or limitations described herein.

The term “dentifrice” as used herein means paste, gel, powder, tablets, or liquid formulations, unless otherwise specified, that are used to clean the surfaces of the oral cavity. Preferably the dentifrice compositions of the present invention are single phase compositions. The term “teeth” as used herein refers to natural teeth as well as artificial teeth or dental prosthesis.

All percentages, parts and ratios are based upon the total weight of the compositions of the present invention, unless otherwise specified. All such weights as they pertain to listed ingredients are based on the active level and, therefore do not include solvents or by-products that may be included in commercially available materials, unless otherwise specified. The term “weight percent” may be denoted as “wt %” herein. All molecular weights as used herein are weight average molecular weights expressed as grams/mole, unless otherwise specified.

As used herein, the articles including “a” and “an” when used in a claim, are understood to mean one or more of what is claimed or described.

As used herein, the terms “comprise”, “comprises”, “comprising”, “include”, “includes”, “including”, “contain”, “contains”, and “containing” are meant to be non-limiting, i.e., other steps and other sections which do not affect the end of result can be added. The above terms encompass the terms “consisting of” and “consisting essentially of”.

As used herein, the words “preferred”, “preferably” and variants refer to embodiments of the invention that afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the invention.

### Ultraphosphate

The present invention is based, in part, of the surprising effectiveness of an ultraphosphate in providing tooth whitening benefits in the dentifrice compositions of the present invention. Ultraphosphate means a condensed phosphate that is generated by polymerizing two or more PO₄ tetrahydras to share an oxygen atom included in other PO₄ tetrahydras. The condensed phosphates are classified into poly(P), metaphosphates and ultraphosphates based on the molar ratio of M₂O/P₂O₅ (wherein “M” represents a monovalent metal typified by an alkali metal). The poly(P) has an M₂O/P₂O₅ molar ratio (R) satisfying 2≧R>1, and is a liner compound represented by Formula (I): Mₘ₊₂PₘO₃ₘ₊₁ (wherein “m” represents an integer of 2 or greater). The metaphosphate has an M₂O/P₂O₅ molar ratio (R) of 1, and is a cyclic or extremely long linear compound represented by Formula (II): (MPO₃)ₙ (wherein “n” represents an integer of 3 or greater). The above-mentioned poly(P) and metaphosphate together are referred to as poly(P). The ultraphosphate has an M₂O/P₂O₅ molar ratio (R) satisfying 1>R>0, and is a compound represented by Formula (III):

$$\frac{x}{2} {M}_{2} O \cdot \frac{y}{2} {P}_{2} {O}_{5}$$

(wherein both x and y represent a positive integer satisfying 0<x/y<1). Ultra-phosphate has a crosslinked mesh-like structure including a branched PO₄ group in the molecule. Commercially available ultraphosphate include sodium ultraphosphate from Mitejima Chemical Co., Ltd (Osaka, Japan). Typical levels of the ultraphosphate in the dentifrice composition of the present invention include from 0.4% to 10%, preferably from 0.4% to 5%, more preferably from 0.4% to 4%, yet more preferably from 0.5% to 3.5%, alternatively from 0.5% to 3%, alternatively from 1% to 3%, by weight of the composition. One aspect of the invention provides for a method for whitening teeth comprising the step of brushing teeth with a dentifrice composition of the present invention.

### Water

The dentifrice compositions of the present invention comprise herein from 45% to 75%, by weight of the composition, of water. Preferably, the dentifrice composition comprises from 45% to 65%, more preferably from 45% to 55%, yet more preferably from 46% to 54%, by weight of the composition, of water. The water may be added to the formulation and/or may come into the composition from the inclusion of other ingredients. Preferably the water is USP water.

### Calcium-containing Abrasive

The compositions of the present invention comprise from 25% to 50%, by weight of the composition, of a calcium-containing abrasive, wherein preferably the calcium-containing abrasive is selected from the group consisting of calcium carbonate, calcium glycerophosphate, dicalcium phosphate, tricalcium phosphate, calcium orthophosphate, calcium metaphosphate, calcium polyphosphate, calcium oxyapatite, sodium carbonate, and combinations thereof. Preferably, the composition comprises from 27% to 47%, more preferably from 27% to 37%, even more preferably from 28% to 34%, yet even more preferably from 29% to 33%, by weight of the composition, alternatively combinations thereof, of a calcium-containing abrasive.

Preferably, the calcium-containing abrasive is calcium carbonate. More preferably, the calcium-containing abrasive is selected from the group consisting of fine ground natural chalk, ground calcium carbonate, precipitated calcium carbonate, and combinations thereof.

Fine ground natural chalk (FGNC) is one of the more preferred calcium-containing abrasives useful in the present invention. It is obtained from limestone or marble. FGNC may also be modified chemically or physically by coating during milling or after milling by heat treatment. Typical coating materials include magnesium stearate or oleate. The morphology of FGNC may also be modified during the milling process by using different milling techniques, for example, ball milling, air-classifier milling or spiral jet milling. One example of natural chalk is described in WO 03/030850 having a medium particle size of 1 to 15 μm and a BET surface area of 0.5 to 3 m²/g. The natural calcium carbonate may have a particle size of 325 to 800 mesh, alternatively a mess selected from 325, 400 600, 800, or combinations thereof; alternatively, the particle size is from 0.1 to 30 microns, or from 0.1 to 20 microns, or from 5 to 20 microns. In one embodiment, the composition comprises from 0% to 5%, preferably 0% to 3%, more preferably 0% to 1%, by weight of the composition, of a silicate; yet more preferably the composition is substantially free silicate.

### Fluoride Ion Source

The compositions may include an effective amount of an anti-caries agent. In one embodiment, the anti-caries agent is a fluoride ion source. The fluoride ion may be present in an amount sufficient to give a fluoride ion concentration in the composition at 25° C., and/or in one embodiment can be used at levels of from 0.0025% to 5% by weight of the composition, alternatively from 0.005% to 2.0% by weight of the composition, to provide anti-caries effectiveness. Representative fluoride ion sources include: stannous fluoride, sodium fluoride, potassium fluoride, amine fluoride, sodium monofluorophosphate, and zinc fluoride. In one embodiment the dentifrice composition contains a fluoride source selected from stannous fluoride, sodium fluoride, and mixtures thereof. In one embodiment, the fluoride ion source is sodium monofluorophosphate, and wherein the composition comprises 0.0025% to 2%, by weight of the composition, of the sodium monofluorophosphate, alternatively from 0.5% to 1.5%, alternatively from 0.6% to 1.7%, alternatively combinations thereof. In another embodiment, the composition comprises from 0.0025% to 2%, by weight of the composition, of a fluoride ion source. In one example, the dentifrice compositions of the present invention may have a dual fluoride ion source, specifically sodium monofluorophosphate and an alkaline metal fluoride. Such an approach may provide an improvement in mean fluoride update.

pH

The pH of the dentifrice composition may be greater than pH 8, preferably greater than pH 8.0, more preferably from pH 8.1 to pH 11. Preferably, the pH is greater than 8.1, more preferably the pH is greater than pH 8.5, even more preferably the pH is greater than pH 9, alternatively the pH is from pH 9.0 to pH 10.5, alternatively from pH 8.5 to pH 10. The relatively high pH of the present inventive composition is for fluoride stability. Without wishing to be bound theory, at below pH 8 calcium ion may bind with the fluoride. Thus, it is desirable to have the dentifrice composition have a greater than pH 8.0 to maximize the stability of the fluoride ion source. A method for assessing pH of dentifrice is described is provided the analytical methods section provided below. For purposes of clarification, although the analytical method describes testing the dentifrice composition when freshly prepared, for purposes of claiming the present invention, the pH may be taken at anytime during the product's reasonable lifecycle (including but not limited to the time the product is purchased from a store and brought to the consumer's home).

### pH Modifying Agent

The dentifrice compositions herein may include an effective amount of a pH modifying agent, alternatively wherein the pH modifying agent is a pH buffering agent. The pH modifying agents, as used herein, refer to agents that can be used to adjust the pH of the dentifrice compositions to the above-identified pH range. The pH modifying agents may include alkali metal hydroxides, ammonium hydroxide, organic ammonium compounds, carbonates, sesquicarbonates, borates, silicates, phosphates, imidazole, and mixtures thereof. Specific pH agents include monosodium phosphate (monobasic sodium phosphate or “MSP”), trisodium phosphate (sodium phosphate tribasic dodecahydrate or “TSP”), sodium benzoate, benzoic acid, sodium hydroxide, potassium hydroxide, alkali metal carbonate salts, sodium carbonate, imidazole, sodium gluconate, lactic acid, sodium lactate, citric acid, sodium citrate, phosphoric acid. In one embodiment, 0.01% to 3%, preferably from 0.1% to 1%, by weight of the composition, of TSP, and 0.001% to 2%, preferably from 0.01% to 0.3%, by weight of the composition, of monosodium phosphate is used. Without wishing to be bound by theory, TSP and monosodium phosphate may also have calcium ion chelating activity and therefore provide some monofluorophosphate stabilization (in those formulations containing monofluorophosphate).

A method for assessing pH of dentifrice is described. The pH is measured by a pH Meter with Automatic Temperature Compensating (ATC) probe. The pH Meter is capable of reading to 0.001 pH unit. The pH electrode may be selected from one of the following (i) Orion Ross Sure-Flow combination: Glass body—VWR #34104-834/Orion #8172BN or VWR#10010-772/Orion #8172BNWP; Epoxy body—VWR #34104-830/Orion #8165BN or VWR#10010-770/Orion #8165BNWP; Semi-micro, epoxy body—VWR #34104-837/Orion #8175BN or VWR#10010-774/Orion #3175BNWP; or (ii) Orion PerpHect combination: VWR #34104-843/Orion #8203BN semi-micro, glass body; or (iii) suitable equivalent. The automatic temperature compensating probe is Fisher Scientific, Cat #13-620-16.

A 25% by weight slurry of dentifrice is prepared with deionized water, and thereafter is centrifuged for 10 minutes at 15000 rotations-per-minute using a SORVALL RC 28S centrifuge and SS-34 rotor (or equivalent gravitational force, at 24149 g force). The pH is assessed in supernatant after one minute or the taking reading is stabilized. After each pH assessment, the electrode is washed with deionized water. Any excess water is wiped with a laboratory grade tissue. When not in issue, the electrode is kept immersed in a pH 7 buffer solution or an appropriate electrode storage solution.

### Low or Free Humectants

The compositions herein may be substantially free or free of humectants, alternatively contain low levels of humectants. The term “humectant,” for the purposes of present invention, include edible polyhydric alcohols such as glycerin, sorbitol, xylitol, butylene glycol, propylene glycol, and combinations thereof. In one embodiment, the humectant is a polyol, preferably wherein the polyol is selected from sorbitol, glycerin, and combinations thereof. In yet another embodiment, the humectant is sorbitol. In one embodiment, the composition comprises from 0% to less than 5%, by weight of the composition, of humectants, preferably from 0% to 4%, alternatively from 0% to 3%, alternatively from 0% to 2%, alternatively from 0% to 1%, by weight of th4 composition, of humectants. A potential advantage of having a dentifrice composition that is free or substantially free of humectants is, without wishing to be bound by theory, is those dentifrice compositions that are free of polyols (e.g., glycerin and sorbitol), or have a relatively low amount thereof, may provide better fluoride uptake compared to those compositions having the high levels of such polyols (or humectants for that matter). In one example, the dentifrice compositions of the present invention comprise from 0% to 5%, preferably 0% to 3%, more preferably 0% to 1%, by weight of the composition, of glycerin, sorbitol, or combinations thereof; yet more preferably the composition is substantially free of both glycerin and sorbitol.

### Thickening System

The dentifrice compositions of the present invention may comprise a thickening system. Preferably the dentifrice composition comprises from 0.5% to 4%, preferably from 0.8% to 3.5%, more preferably from 1% to 3%, yet still more preferably from 1.3% to 2.6%, by weight of the composition, of the thickening system. More preferably the thickening system comprises a thickening polymer, a thickening silica, or a combination thereof. Yet more preferably, when the thickening system comprises a thickening polymer, the thickening polymer is selected from a carboxymethyl cellulose, a linear sulfated polysaccharide, a natural gum, and combination thereof. Yet still more preferably, when the thickening system comprises a thickening polymer, the thickening polymer is selected from the group consisting of: (a) 0.01% to 3% of a carboxymethyl cellulose (“CMC”) by weight of the composition, preferably 0.1% to 2.5%, more preferably 0.2% to 1.5%, by weight of the composition, of CMC; (b) 0.01% to 2.5%, preferably 0.05% to 2%, more preferably 0.1% to 1.5%, by weight of the composition, of a linear sulfated polysaccharide, preferably wherein the linear sulfated polysaccharide is a carrageenan; (c) 0.01% to 7%, preferably 0.1% to 4%, more preferably from 0.1% to 2%, yet more preferably from 0.2% to 1.8%, by weight of the composition, of a natural gum; (d) combinations thereof. Preferably when the thickening system comprises a thickening silica, the thickening silica is from 0.01% to 10%, more preferably from 0.1% to 9%, yet more preferably 1% to 8% by weight of the composition.

Preferably the linear sulfated polysaccharide is a carrageenan (also known as carrageenin). Examples of carrageenan include Kappa-carrageenan, Iota-carrageenan, Lambda-carrageenan, and combinations thereof.

In one example the thickening silica is obtained from sodium silicate solution by destabilizing with acid as to yield very fine particles. One commercially available example is ZEODENT® branded silicas from Huber Engineered Materials (e.g., ZEODENT® 103, 124, 113 115, 163, 165, 167).

In one example the CMC is prepared from cellulose by treatment with alkali and monochloro-acetic acid or its sodium salt. Different varieties are commercially characterized by viscosity. One commercially available example is Aqualon™ branded CMC from Ashland Special Ingredients (e.g., Aqualon™ 7H3SF; Aqualon™ 9M3SF Aqualon™ TM9A; Aqualon™ TM12A).

Preferably a natural gum is selected from the group consisting of gum karaya, gum arabic (also known as acacia gum), gum tragacanth, xanthan gum, and combination thereof. More preferably the natural gum is xanthan gum. Xanthan gum is a polysaccharide secreted by the bacterium Xanthomonas camestris. Generally, xanthan gum is composed of a pentasaccharide repeat units, comprising glucose, mannose, and glucuronic acid in a molar ratio of 2:2:1, respectively. The chemical formula (of the monomer) is C₃₅H₄₉O₂₉. In one example, the xanthan gum is from CP Kelco Inc (Okmulgee, US).

### Viscosity

Preferably the dentifrice compositions of the present invention have a viscosity range from 150,000 centipoise to 850,000 centipoise (“cP”). A method for assessing viscosity is described. The viscometer is Brookfield® viscometer, Model DV-I Prime with a Brookfield “Helipath” stand. The viscometer is placed on the Helipath stand and leveled via spirit levels. The E spindle is attached, and the viscometer is set to 2.5 RPM. Detach the spindle, zero the viscometer and install the E spindle. Then, lower the spindle until the crosspiece is partially submerged in the paste before starting the measurement. Simultaneously turn on the power switch on the viscometer and the helipath to start rotation of the spindle downward. Set a timer for 48 seconds and turn the timer on at the same time as the motor and helipath. Take a reading after the 48 seconds. The reading is in cP.

### PEG

The compositions of the present invention may optionally comprise polyethylene glycol (PEG), of various weight percentages of the composition as well as various ranges of average molecular weights. In one aspect of the invention, the compositions have from 0.01% to 8%, preferably from 0.1% to 5%, more preferably from 0.2% to 4.8%, yet more preferably from 0.3% to 4.2%, yet still more preferably from 0.5% to 4%, by weight of the composition, of PEG. In another aspect of the invention, the PEG is one having a range of average molecular weight from 100 Daltons to 1600 Daltons, preferably from 200 to 1000, alternatively from 400 to 800, alternatively from 500 to 700 Daltons, alternatively combinations thereof. PEG is a water soluble linear polymer formed by the addition reaction of ethylene oxide to an ethylene glycol equivalent having the general formula is: H—(OCH₂CH₂)ₙ—OH. One supplier of PEG is Dow Chemical Company under the brandname of CARBOWAX™. Without wishing to be bound by theory, having some PEG in the dentifrice composition may help with physical stability.

### Sweetener

The oral care compositions herein may include a sweetening agent. These include sweetening agents may include saccharin, dextrose, sucrose, lactose, maltose, levulose, aspartame, sodium cyclamate, D-tryptophan, dihydrochalcones, acesulfame, sucralose, neotame, and mixtures thereof. Sweetening agents are generally used in oral compositions at levels of from 0.005% to 5%, by weight of the composition, alternatively 0.01% to 1%, alternatively from 0.1% to 0.5%, alternatively combinations thereof.

### Surfactant

The dentifrice compositions herein may include a surfactant. The surfactant may be selected from anionic, nonionic, amphoteric, zwitterionic, cationic surfactants, or mixtures thereof. The composition may include a surfactant at a level of from 0.1% to 10%, from 0.025% to 9%, from 0.05% to 5%, from 0.1% to 2.5%, from 0.5% to 2%, or from 0.1% to 1% by weight of the total composition. Non-limiting examples of anionic surfactants may include those described at US 2012/0082630 A1 at paragraphs 32, 33, 34, and 35. Non-limiting examples of zwitterionic or amphoteric surfactants may include those described at US 2012/0082630 A1 at paragraph 36; cationic surfactants may include those described at paragraphs 37 of the reference; and nonionic surfactants may include those described at paragraph 38 of the reference. In one embodiment the composition comprises 0.1% to 5%, preferably 0.1% to 3%, alternatively from 0.3% to 3%, alternatively from 1.2% to 2.4%, alternatively from 1.2% to 1.8%, alternatively from 1.5% to 1.8%, by weight of the composition, alternatively combinations thereof, of the anionic surfactant sodium lauryl sulfate (SLS).

### Colorant

The compositions herein may include a colorant. Titanium dioxide is one example of a colorant. Titanium dioxide is a white powder which adds opacity to the compositions. Titanium dioxide generally can comprise from 0.25% to 5%, by weight of the composition.

### Flavorant

The compositions herein may include from 0.001% to 5%, alternatively from 0.01% to 4%, alternatively from 0.1% to 3%, alternatively from 0.5% to 2%, alternatively 1% to 1.5%, alternatively 0.5% to 1%, by weight of the composition, alternatively combinations thereof, of a flavorant composition. The term flavorant composition is used in the broadest sense to include flavor ingredients, or sensates, or sensate agents, or combinations thereof. Flavor ingredients may include those described in US 2012/0082630 A1 at paragraph 39; and sensates and sensate ingredients may include those described at paragraphs 40-45, incorporated herein by reference. Excluded from the definition of flavorant composition is “sweetener” (as described above).

### EXAMPLES

Tooth stain prevention and tooth stain removal is assessed for an inventive dentifrice composition and comparative compositions as well as a commercial product. Analytical techniques, composition components (of examples 1-6), and results are described.

The hydroxyapatite (HAP) powder whitening model analytical technique is described. Briefly, this technique is a way to evaluate chemical actions of dentifrice components in the non-abrasive removal and prevention of a tea stain. In this technique, the main component of human teeth (i.e,. HAP) is used as tea stain absorption substrate to perform an in-vitro test. Pre-treatment with dentifrice reduces tea adsorption to powdered apatite, while post treatments of pre-stained powder results in desorption of tea components. Visual comparison and color reading with colorimeter are applied to conduct qualitative and quantitative evaluations.

Materials and methods are described. Materials include hydroxyapatite (Wako); black tea (Lipton); filtration paper (Whatman); and 1 N HCL. Black tea is prepared by adding four tea bags in 100 ml of boiling water for 30 minutes. The tea solution is used within 30 minutes after filtration. Dentifrice/water slurry (1:3 weight ratio) is prepared by weighing 5 grams of dentifrice and 15 grams of water in a centrifuge tube and mechanically stirring. Thereafter, the slurry is centrifuged at 15,000 rotations per minute (RPM) for 15 minutes. The resulting supernatant (“Dentifrice Slurry”) is removed for later use.

HAP powder preparation is described. 0.4 g of HAP powder is placed in a centrifuge tube. 10 ml is added to the tube and then the tube is vortexed for 3 minutes. The vortexed tube is then centrifuged at 15,000 RPM for 15 minutes, thereafrer the supernatant is discarded. Add 20m1 water is added and then the tub is vortexed for 2 minutes, and thereafter the tube is centrifuge at 15,000 RPM for 15 minutes discarding the resulting supernatant. The aforementioned step is repeated another two times. 10 ml of the Dentifrice Slurry is added to the tub and then vortexed for one minute, and thereafter the tube is centrifuged 15000 RPM for 15 minutes discarding the resulting supernatant. 20 ml water is added to the tub and vortex for two minutes, and thereafter the tube is centrifuged at 15000 RPMS for 15 minutes discarding the resulting supernatant. 20 ml of water is added to the tube and then vortexing the tub for two minutes to provide a dispersion. The dispersion is filtered with filtration paper. The resulting powder is washed with 10 ml of deionized water. The washed powder is formed into a powder disc and is dried at room temperature overnight. The dried powder disc is ready for visual comparison and quantitative analysis.

A high efficacy control (“Control H”) and a low efficacy control (“Control L”) are prepared. Tea and Dentifrice Slurry are replaced with water to prepare Control H while water is replaces the Dentifrice Slurry to prepare the Control L. The procedure for stain prevention and stain treatment are generally the same except the order of addition of the Dentifrice Slurry and the black tea.

Qualitative and quantative evaluation is described. The whitening efficacy of the dentifrice examples and commercial product are evaluated by visually comparing the color of treated HAP powder disc. That is, the darker the color of the disc, the lower the efficacy of the example or commercial product. Quantitative evaluation is conducted by color reading and analysis. Color is evaluated from the basic color elements L (light/dark), a (red/green) and b (yellow/blue). The color difference of one sample from standard is evaluated by ΔL, Δa and Δb. An integrated parameter ΔE is used to conduct a general comparison, where a greater ΔE value means a lower whitening efficacy of corresponding example/product.

Ultraviolet (UV) analysis is described. The premise of the UV analysis to assess whitening efficacy is based upon principle that if one solute in a solution has absorption on UV spectrometer, the absorbance reflects the amount of the subject solute in the solution. By scanning black tea with a UV spectrophotometer, black tea is observed as having strong absorbance at 350 nm. Accordingly, this UV test is applied to compare the color of treated HAP powder discs to evaluate the efficacy of different dentifrice examples/product on stain prevention and stain removal. In this UV experiment, the treated HAP powder disc is dissolved in 1N HCl before recording the solution's absorbance at 350 nm. Specifically, 0.2 grams of dry treated HAP powder is placed in a 10 ml volume flask and 1N HCl is added to volume. The flaks is shaken until the powder is completed dissolved. The dissolved solution is centrifuged at 15,000 RPM for ten minutes. The absorbance of the resulting supernatant is measured at 350 nm with a UV spectrophotomer. Deionized water is used as a reference and 1 N HC1 is used as a blank. The lower the absorbance, the higher the whitening efficacy is the subject example/product. To enable comparison of data generated between different experiments, a relative Efficacy Value P is employed. The definition of P is described: P=(A_{L}−A_{dentifrice})/(A_{L}−A_{H})*100; wherein “A” is the value of UV absorbance at 400 nm; “A_{L}” is absorbance of L control; and “A_{H}” is absorbance of high efficacy control.

TABLE 1
Phosphate containing agents are provided including the molecular weight,
phosphate content, and the amount of agent added in the formulations
with corresponding weight percentage in the subject example.

| | | | Phosphate | |
|---|---|---|---|---|
| | | | Content | |
| Phosphate Reagents | Formula | Mol Wt | Added | w/w % |
| Tetrasodium Pyrophosphate | Na₄O₇P₂ | 266 | 1.30 | 5.56 |
| (TSPP) | | | | |
| Na Ultraphosphate | Na₃O₁₀P₆ | 321.88 | 1.30 | 2.25 |
| Ca Glycerol phosphate | C₃H₇CaO₆P | 210 | 1.30 | 8.78 |
| Na Acid Pyrophosphate | Na₂H₂P₂O₇ | 222 | 1.30 | 4.65 |
| (SAPP) | | | | |

TABLE 2
Components (on a weight percentage basis) are provided for
inventive example 4 and comparative examples 1, 2, 3, and
5, 6. Notably, example 4 contains sodium ultraphosphate.
The other comparative examples contain
phosphate agents indicated in table 1 above.

| Components | | | | Ex. 4 | | |
|---|---|---|---|---|---|---|
| (weight percent) | Ex. 1 | Ex. 2 | Ex. 3 | (Inv.) | Ex. 5 | Ex. 6 |
| Tetra Sodium | 0 | 5.56 | 5.56 | — | | |
| Pyrophosphate | | | | | | |
| Sodium | — | — | 9 | — | — | — |
| Bicarbonate | | | | | | |
| Calcium | — | — | 0.2 | — | — | — |
| Peroxide | | | | | | |
| Na | — | — | — | 2.25 | — | — |
| Ultraphosphate | | | | | | |
| Calcium | — | — | — | — | 8.78 | — |
| Glycero- | | | | | | |
| phosphate | | | | | | |
| Sodium Acid | | | | | | 4.65 |
| Pyrophosphate | | | | | | |
| Carrageenan | 1.2 | 1.2 | 1.2 | 1.2 | 1.2 | 1.2 |
| Sodium | 0.91 | 0.91 | 0.91 | 0.91 | 0.91 | 0.91 |
| Carboxymethyl | | | | | | |
| Cellulose | | | | | | |
| Zeodent 165 | 2.62 | 2.62 | 2.62 | 2.62 | 2.62 | 2.62 |
| Sodium | 0.25 | 0.25 | 0.25 | 0.25 | 0.25 | 0.25 |
| Saccharin | | | | | | |
| Sodium | 1.1 | 1.1 | 1.1 | 1.1 | 1.1 | 1.1 |
| Monofluoro- | | | | | | |
| phosphate | | | | | | |
| Sodium | 0.08 | 0.08 | 0.08 | 0.08 | 0.08 | 0.08 |
| Monophosphate | | | | | | |
| Sodium | 0.42 | 0.42 | 0.42 | 0.42 | 0.42 | 0.42 |
| Triphosphate | | | | | | |
| Calcium | 32 | 32 | 32 | 32 | 32 | 32 |
| Carbonate | | | | | | |
| Sodium Lauryl | 4 | 4 | 4 | 4 | 4 | 4 |
| Sulfate | | | | | | |
| Flavor | 0.85 | 0.85 | 0.85 | 0.85 | 0.85 | 0.85 |
| Deionized | 56.57 | 51.01 | 41.81 | 54.32 | 47.79 | 51.92 |
| Water | | | | | | |
| Total | 100.0 | 100.0 | 100.0 | 100.0 | 100.0 | 100.0 |

Data is provided regarding the above-identified examples. Examples 1-6, having compositional components described in Table 2, are assessed for stain prevention and stain removal according the method previously described. A silica-based commercial product is also assessed. Table 3 below provides for each of the examples: pH, and stain prevention and the stain removal values.

TABLE 3
Stain Prevention and Stain Removal of subject examples
and commercialized product are provided.

| Example/ | | Stain | Stain | |
|---|---|---|---|---|
| Product | Notable Components | Prevention | Removal | pH |
| Ex. 1 | Nil TSPP | 14.36 | 16.50 | 8.57 |
| Ex. 2 | 5.56 wt % TSPP | 26.43 | 58.18 | 9.40 |
| Ex. 3 | 9 wt % Baking Soda; | 22.65 | 59.66 | 9.30 |
| | 0.2 wt % Ca Peroxide; | | | |
| | 5.56 wt % TSPP | | | |
| Ex. 4 | 2.25 wt % Na | 94.93 | 55.64 | 8.96 |
| (Inventive) | Ultraphosphate | | | |
| Ex. 5 | 8.78 wt % Calcium | 14.63 | 19.70 | 8.52 |
| | Glycerophosphate | | | |
| Ex 6 | 4.65 wt % SAPP | 30.01 | 24.41 | 7.76 |
| DARLIE ® | Silica; | 58.02 | 57.57 | 7.7 |
| Super White¹ | ~5% TSPP | | | |

¹Lot#: EXP2015041

As indicated in Table 3, inventive example 4 had the greatest stain prevention value as compared to comparative examples and even the commercialized product marketing a tooth whitening benefit. The phosphate content for each of the examples/products is essentially the same.

Further stain prevention and stain removal data is provided. An unaided visual assessment is made of the examples and a commercial product by comparing the stain prevention and stain removal by visualizing comparing the treated HAP powder discs compared to each other and the positive and negative controls. The method is followed is previously described. A seven point “+” scale for stain prevention and a five point “+” scale for stain removal are used to represent high efficacy control wherein a low efficacy control represent a single “+.” Examples and product are assessed in this context. An example or product closer to the high efficacy control (i.e., Control H) is more desirable from a tooth whitening perspective. Results are provided in Table 4 below.

TABLE 4
Stain prevention and removal are assessed in example 1-6
and commercialized product.

| Example/ | | | |
|---|---|---|---|
| Product | Notable Components | Stain Prevention | Stain Removal |
| Control H | High Efficacy Control | +++++++ | + ++++ |
| Control L | Low Efficacy Control | + | + |
| Ex. 1 | Nil TSPP | ++ | ++ |
| Ex. 2 | 5.56 wt % TSPP | +++ | +++ |
| Ex. 3 | 9 wt % Baking Soda; | +++ | +++ |
| | 0.2 wt % Ca Peroxide; | | |
| | 5.56 wt % TSPP | | |
| Ex. 4 | 2.25 wt % Na | +++++++ | +++ |
| (Inventive) | Ultraphosphate | | |
| Ex. 5 | 8.78 wt % Calcium | ++ | + + |
| | Glycerophosphate | | |
| Ex 6 | 4.65 wt % SAPP | +++ | + + |
| DARLIE ® | Silica; | ++++ | +++ |
| Super White¹ | ~5% TSPP | | |

¹Lot#: EXP2015041

Table 4 indicates that inventive example 4 provides the greatest results in both stain removal and stain prevention.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A dentifrice composition comprising:
(a) 45% to 75%, by weight of the composition, of water;
(b) 25% to 50%, by weight of the composition, of a calcium-containing abrasive;
(c) 0.0025% to 2%, by weight of the composition, of a fluoride ion source;
(d) 0.4% to 10%, by weight of the composition, of an ultraphosphate; and
wherein said composition has a pH greater than 8.

2. The dentifrice composition of claim 1, wherein dentifrice composition comprises from 0.4% to 5%, by weight of the composition, of the ultraphosphate.

3. The dentifrice composition of claim 2, wherein dentifrice composition comprises from 0.5% to 3.5%, by weight of the composition, of the ultraphosphate.

4. The dentifrice composition of claim 1, wherein the ultraphosphate is made by polymerizing two more PO₄ tetrahydras to share an oxygen atom included in other PO₄ tetrahydras.

5. The dentifrice composition of any one of claim 1, wherein the composition comprises from 0.2% to 1.4% fluoride ion source and wherein the fluoride ion source comprises sodium monofluorophosphate.

6. The dentifrice composition of any one of claim 1, wherein the composition comprises from 0.5% to 1% fluoride ion source and wherein the fluoride ion source comprises sodium monofluorophosphate.

7. The dentifrice composition of claim 6, wherein the composition comprises from 27% to 37%, by weight of the composition, of the calcium carbonate wherein the calcium-containing abrasive comprises calcium carbonate.

8. The dentifrice composition of claim 1, wherein the pH is greater than pH 8.5.

9. The dentifrice composition of claim 7 wherein the pH is from 8.5 to 10.5.

10. The dentifrice composition of claim 1, wherein the composition comprises from 50% to 60%, by weight of the composition, of water.

11. The dentifrice composition of claim 1, wherein the composition is a single phase composition.

12. The dentifrice composition of claim 1, wherein the composition comprises from 0% to 5%, by weight of the composition, of a humectant selected from the group consisting of glycerin, sorbitol, or combinations thereof.

13. The dentifrice composition of claim 1, wherein the composition is substantially free of glycerin and sorbitol.

14. The dentifrice composition of claim 1, wherein the composition comprises from 0% to 5%, by weight of the composition, of a silicate.

15. The dentifrice composition of claim 13, wherein the composition is substantially free of the silicate.

16. The dentifrice composition according to claim 1, further comprising a thickening system, wherein the thickening system is selected from the group consisting of a thickening polymer, a thickening silica, or combinations thereof.

17. The dentifrice composition according to claim 15, wherein the thickening system comprises a thickening polymer wherein the thickening polymer is selected from the group consisting of carboxymethyl cellulose, linear sulfated polysaccharide, natural gum, and combinations thereof.

18. The dentifrice composition according to claim 16, wherein the thickening polymer comprises from 0.01% to 3%, by weight of the composition, carboxymethyl cellulose.

19. The dentifrice composition according to claim 16, wherein the thickening polymer comprises from 0.01% to 2.5%, by weight of the composition, linear sulfated polysaccharide.

20. The dentifrice composition according to claim 18, wherein the linear sulfated polysaccharide comprises carrageenan.

21. A method of preventing or removing a stain from teeth comprising the step of brushing teeth with the dentifrice composition claim 1.
